(19) United States
(12) Patent Application Publication
(10) Pub. No.: US 2024/0091790 A1
(43) Pub. Date: Mar. 21, 2024

(54) EXTERNALLY-MOUNTED AND DETACHABLE AIR PURIFICATION LAMP FITTING

(71) Applicant: Yanhua CHEN, Shenzhen (CN)

(72) Inventor: Yanhua CHEN, Shenzhen (CN)

(21) Appl. No.: 18/110,485

(22) Filed: Feb. 16, 2023

(30) Foreign Application Priority Data
Sep. 17, 2022 (CN) 2022224612122

(51) Int. Cl. F21V 33/00; A61L 9/22; B01D 46/00; B01D 46/24; B03C 3/155; B03C 3/36

(52) U.S. Cl. CPC F21V 33/0096; A61L 9/22; B01D 46/0028; B01D 46/24; B03C 3/155; B03C 3/368; A61L 2209/134; A61L 2209/14; A61L 2209/15; B01D 2279/65

(57) ABSTRACT

An externally-mounted and removable air purification lamp fitting includes a fitting structure arranged on a lamp, the fitting structure includes a breathable outer cover with an opening at its bottom, a side wall of the breathable outer cover is evenly provided with breathable through holes, a top of the breathable outer cover is provided with an air guide hole, a lower part of the breathable outer cover is fixed with a bottom cover, the breathable outer cover is provided with a formaldehyde removal filter cover therein, an inner side of the formaldehyde removal filter cover is provided with an exhaust fan and an anion generator, and negative ions generated by the anion generator and purified air are diffused to outside through the exhaust fan. The formaldehyde removal filter cover cooperates with the exhaust fan and the anion generator, which can effectively remove dust and improve air purification performance.

### TECHNICAL FIELD

The disclosure belongs to the technical field of air purification lamps, in particular to an externally-mounted and detachable air purification lamp fitting.

### BACKGROUND

With the rapid development of the industrial technology, the air pollution is more and more serious, and the polluted weather brings a certain influence to the life of people, so in order to solve the problem of the air pollution, the existing air purification lamp has more designs, for example, Chinese patent application with the application number of 201621158286.7, titled a double-filtering air purification lamp belongs to the design of loading an air purification device on a lamp. However, a plurality of air purification lamps have a larger defect that the air purification lamps are loaded with the air purification device, a related filtering system of the air purification device needs to be regularly replaced and cleaned, and the current air purification lamp is troublesome to clean and replace, the lamp needs to be integrally disassembled by means of a ladder and then cleaned and replaced, and the process is quite complicated. Moreover, the disassembly and assembly operation is not convenient, which will affect its scope of application to a certain extent and limit its practicability.

### SUMMARY

The disclosure aims to provide an externally-mounted and detachable air purification lamp fitting with reasonable structure and convenient disassembly, assembly and maintenance.

The technical scheme for realizing the purpose of the disclosure is that an externally-mounted and detachable air purification lamp fitting includes a fitting structure arranged on a lamp, where the fitting structure includes a breathable outer cover with an opening at the bottom; a side wall of the breathable outer cover is evenly provided with breathable through holes; a top of the breathable outer cover is provided with an air guide hole; the bottom of the breathable outer cover is provided with a bottom cover; a formaldehyde removal filter cover is disposed inside the breathable outer cover; an exhaust fan and an anion generator are disposed inside the formaldehyde removal filter cover; and negative ions generated by the anion generator and purified air are diffused to outside through the exhaust fan.

In an embodiment, an external light emitting module is fixed on a bottom surface of the bottom cover, and a light emitting source is fixed in the external light emitting module.

In an embodiment, the top of the breathable outer cover is provided with a mounting circular hole for fixing and mounting.

In an embodiment, the formaldehyde removal filter cover is annular, an outer diameter of the formaldehyde removal filter cover is the same as an inner diameter of the breathable outer cover, an outer surface of the formaldehyde removal filter cover is attached to an inner wall of the breathable outer cover, and the exhaust fan and the anion generator are located in an inner ring of the formaldehyde removal filter cover.

In an embodiment, the lamp is one of a ceiling lamp, a pendant lamp and a floor lamp.

In an embodiment, the breathable outer cover is a cylindrical cover, and the formaldehyde removing filter cover is a circular ring cover.

The disclosure has positive effects that the structure of the externally-mounted and detachable air purification lamp fitting provided by the disclosure is reasonable, the formaldehyde removal filter cover is matched with the exhaust fan and the anion generator, so that dust removal, sterilization and purification operations can be effectively carried out on the air, and the air purification performance is improved; in addition, the externally-mounted and detachable air purification lamp fitting provided by the disclosure is an independent structure arranged on the lamp, so that disassembly, assembly, overhaul and maintenance operations can be conveniently carried out. The application convenience and that application range are improve, and the practicability is strong; and moreover, an external light emitting module can be additionally arranged, so that the lamp has the air purification effect of removing dust, removing formaldehyde from air and the like, and also has the lighting performance, so that the practicability is strong.

### BRIEF DESCRIPTION OF THE DRAWINGS

In order that the contents of the disclosure may be more clearly understood, the disclosure will be described in further detail with reference to specific embodiments and with reference to the accompanying drawings, in which:

FIG. 1 is a schematic structural diagram of an externally-mounted and detachable air purification lamp fitting according to embodiments of the disclosure.

FIG. 2 is another schematic structural diagram of an externally-mounted and detachable air purification lamp fitting according to embodiments of the disclosure.

FIG. 3 is a schematic diagram of an externally-mounted and detachable air purification lamp fitting when it is applied to a floor lamp according to embodiments of the disclosure.

FIG. 4 is a partial structure diagram of the externally-mounted and detachable air purification lamp fitting as shown in FIG. 3.

FIG. 5 is a schematic diagram of an externally-mounted and detachable air purification lamp fitting when it is applied to a ceiling light according to embodiments of the disclosure.

FIG. 6 is a schematic diagram of the externally-mounted and detachable air purification lamp fitting as shown in FIG. 5 from another perspective.

FIG. 7 is a schematic diagram of an externally-mounted and detachable air purification lamp fitting when it is applied to a table lamp according to embodiments of the disclosure.

FIG. 8 is a schematic structural diagram of a breathable outer cover of the externally-mounted and detachable air purification lamp fitting according to embodiments of the disclosure.

### DESCRIPTION OF REFERENCE NUMERALS

1: lamp, 2: fitting structure; 21: breathable outer cover; 22: breathable through hole; 23: air guide hole; 24: bottom cover; 25: formaldehyde removal filter cover; 26: exhaust fan; 27: anion generator; 3: mounting circular hole; 4: external light emitting module.

### DETAILED DESCRIPTION

### Embodiment 1

The technical schemes in the embodiments of the disclosure will be described clearly and completely below in combination with the drawings in the embodiments of the disclosure. Apparently, the described embodiments are only part of the embodiments of the disclosure, not all of them. Based on the embodiments of the disclosure, all other embodiments obtained by those skilled in the art without doing creative work belong to the scope of protection of the disclosure.

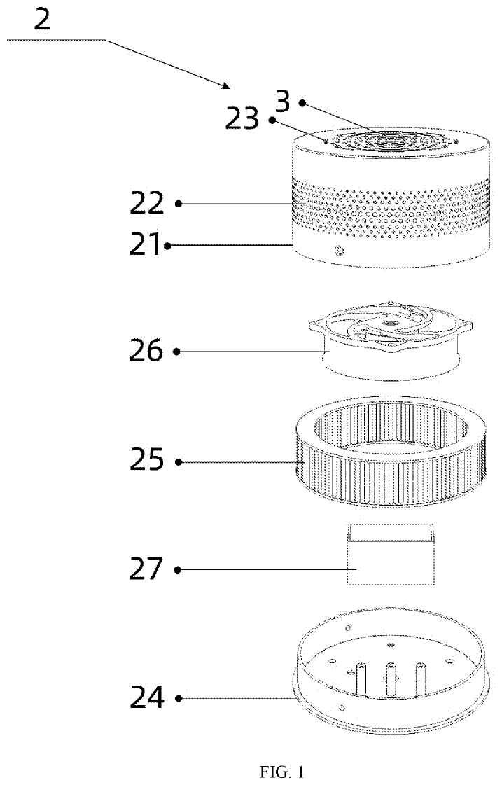

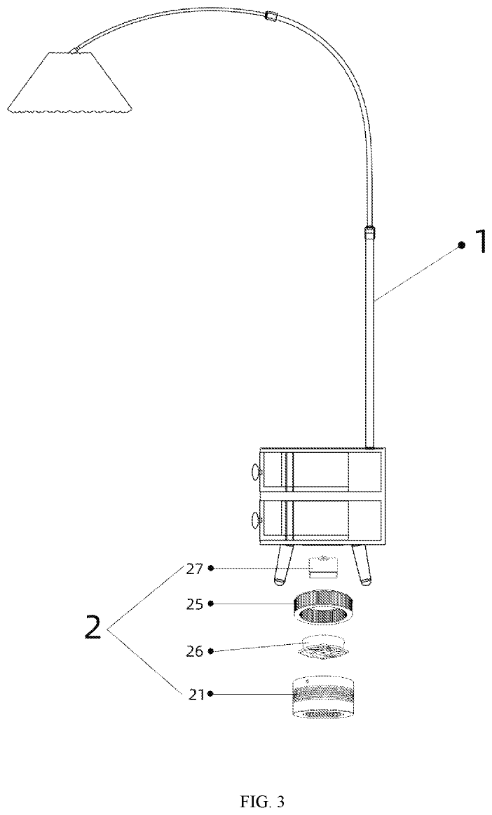

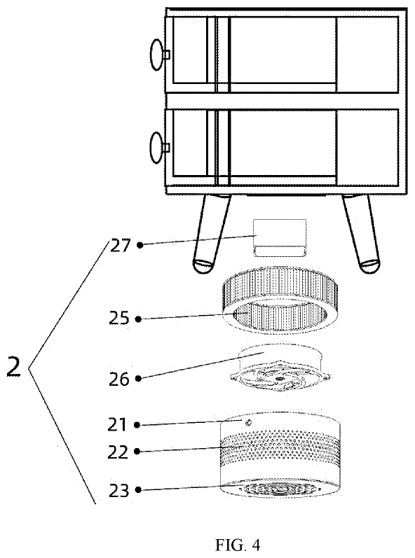

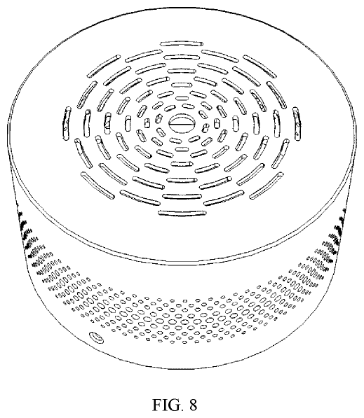

As shown in FIG. 1, FIG. 3, FIG. 4 and FIG. 8, an externally-mounted and detachable air purification lamp fitting includes a fitting structure 2 arranged on a lamp 1, and the fitting structure 2 includes a breathable outer cover 21 with an opening at its bottom. In this embodiment, the breathable outer cover 21 can be a plastic structure or a metal structure, and its shape can be cylindrical or square.

A side wall of the breathable outer cover is evenly provided with breathable through holes 22, and a top of the breathable outer cover is provided with an air guide hole 23, the externally-mounted and detachable air purification lamp fitting is provided with the breathable through holes 22 to match with the air guide hole 23, so that external air can circulate through the breathable through holes 22 and the air guide hole 23 under the action of an exhaust fan (i.e., air diffuser fan) 26, and dust removal, sterilization and purification are carried out through an internal formaldehyde removing filter cover 25 and an anion generator (i.e., negative ion generator) 27, thus ensuring the quality of the dispersed air.

A bottom cover 24 is fixed at a lower part of the breathable outer cover 21, a formaldehyde removing filter cover 25 is arranged inside the breathable outer cover 21, and an exhaust fan 26 and an anion generator 27 are fixed at the inner side of the formaldehyde removing filter cover 25, and the anion (i.e., negative ions) generated by the anion generator 27 and purified air are diffused to the outside through the exhaust fan 26. In this embodiment, the bottom cover 24 is matched with the breathable outer cover 21 to form a cavity inside, which is also beneficial to positioning and fixing the internal structure. In the actual application process, the bottom cover 24 can also be provided with a power supply structure such as a battery, and the exhaust fan 26 and the anion generator 27 are of the prior art.

The anion generator 27 will release negative ions, which can be used as an action factor to actively capture harmful substances in the passing air and purify the air by dust removal and sterilization.

The formaldehyde removing filter cover 25 can perform formaldehyde removal and dust removal operations on the air passing through the breathable through holes 22, so as to achieve primary purification, and the formaldehyde removing filter cover 25 cooperates with the anion generator 27 to perform secondary purification on the air, so as to improve the overall air purification quality.

The exhaust fan 26 mainly diffuses the negative ions generated by the anion generator 27 and purified air to the outside.

In the actual application process, the externally-mounted and detachable air purification lamp fitting of the embodiment needs to be installed on the lamp, so its power supply is consistent with that of the lamp, and it can be directly connected with the lamp through conductive wires.

In the actual application process, the top of the breathable outer cover is provided with a mounting circular hole 3 for fixed mounting. It can be connected and used through the mounting circular hole 3 as a fixture of a pendant lamp, which can not only ensure the stability and reliability of the connection, but also realize conductive connection. At the same time, it is convenient to disassemble and assemble.

And in the actual application process, the formaldehyde removal filter cover is annular, an outer diameter of the formaldehyde removal filter cover is the same as an inner diameter of the breathable outer cover, an outer surface of the formaldehyde removal filter cover is attached to an inner wall of the breathable outer cover, and the exhaust fan 26 and the anion generator 27 are located in an inner ring of the formaldehyde removal filter cover 25. The shape of the formaldehyde removal filter cover 25 is the same as that of the breathable outer cover 21. When the breathable outer cover 21 is cylindrical, the formaldehyde removal filter cover 25 is circular, and when the breathable outer cover 21 is square, the formaldehyde removal filter cover 25 is square.

In this embodiment, the lamp is a floor lamp, which can be a conventional floor lamp structure such as a desk lamp. The externally-mounted and detachable air purification lamp fitting is located at a bottom of a lamp base of the floor lamp.

The disclosure has the positive effects that the structure of the disclosure is reasonable, the formaldehyde removal filter cover is provided with the exhaust fan and the anion generator, which can effectively remove dust, sterilize and purify the air, and improve the air purification performance; Moreover, the disclosure is a separate structure arranged on the lamp, which is convenient for disassembly, overhaul and maintenance, thus improving the application convenience and scope, and having strong practicability; Moreover, it can be additionally provided with an external light emitting module, so that it has lighting performance besides air purification effects such as dust removal and air in addition to formaldehyde, and has strong practicability.

### Embodiment 2

The technical schemes in the embodiments of the disclosure will be described clearly and completely below in combination with the drawings in the embodiments of the disclosure. Apparently, the described embodiments are only part of the embodiments of the disclosure, not all of them. Based on the embodiments of the disclosure, all other embodiments obtained by those skilled in the art without doing creative work belong to the scope of protection of the disclosure.

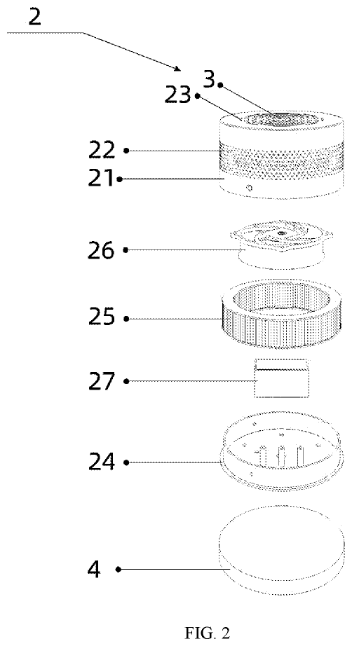

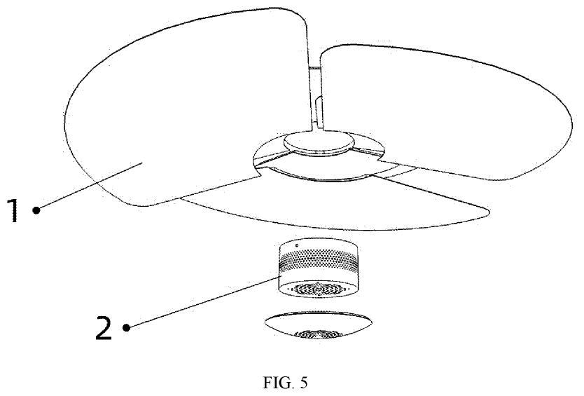

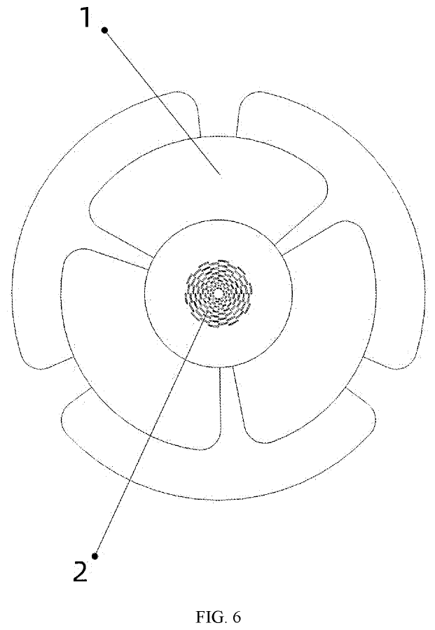

As shown in FIG. 2, FIG. 5 and FIG. 6, this embodiment is basically the same as Embodiment 1, but the difference is that the bottom surface of the bottom cover is fixed with an external light emitting module 4, and the external light emitting module 4 is fixed with a light source therein. In this embodiment, the external light emitting module can be added, the external light source plate can be replaced for lighting, and the power supply of the external light emitting module is the same as that of the lamp. The power supply can be connected through the power line, and the lighting function can also be provided after the purification functions such as dust removal, formaldehyde removal and sterilization, which can meet the use needs of different situations.

In this embodiment, the lamp is a ceiling lamp. In the actual application process, the lamp can also be a pendant lamp or other lighting lamp structure. The fitting structure is at the bottom center of the lamp.

### Embodiment 3

The technical schemes in the embodiments of the disclosure will be described clearly and completely below in combination with the drawings in the embodiments of the disclosure. Apparently, the described embodiments are only part of the embodiments of the disclosure, not all of them. Based on the embodiments of the disclosure, all other embodiments obtained by those skilled in the art without doing creative work belong to the scope of protection of the disclosure.

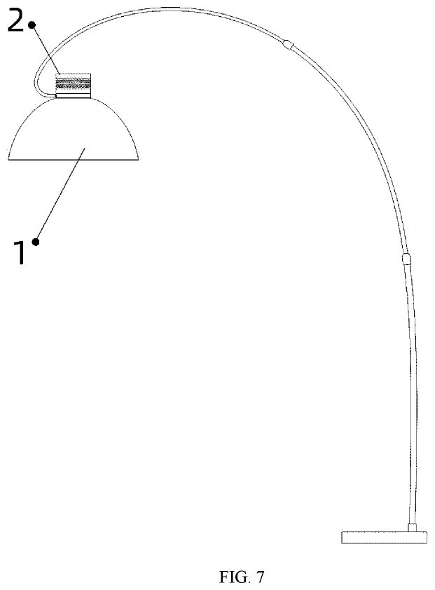

As shown in FIG. 1 and FIG. 7, this embodiment is basically the same as Embodiment 1, but the difference is that in this embodiment, the lamp is a desk lamp structure, and its fitting structure is located on an upper part of a desk lamp cover.

The standard parts used in these embodiments can be directly purchased from the market, and the non-standard structural components recorded in the specification can also be directly processed according to the existing technical general knowledge without doubt. At the same time, the connection mode of each component adopts the mature conventional means in the existing technology, while the machinery, parts and equipment all adopt the conventional models in the existing technology, so no specific description will be made here.

Apparently, the above embodiments of the disclosure are only to clearly illustrate the examples of the disclosure, not to limit the implementation mode of the disclosure. For those skilled in the art, other changes or changes in different forms can be made on the basis of the above description. It is unnecessary and impossible to enumerate all embodiments here. However, these obvious changes or changes caused by the essential spirit of the disclosure still belong to the protection scope of the disclosure.

I/We claim:

1. An externally-mounted and detachable air purification lamp fitting, comprising: a fitting structure disposed on a lamp; wherein the fitting structure comprises: a breathable outer cover with an opening at a bottom, a side wall of the breathable outer cover is evenly provided with breathable through holes, a top of the breathable outer cover is provided with an air guide hole, the bottom of the breather outer cover is fixedly provided with a bottom cover, a formaldehyde removing filter cover is disposed inside the breathable outer cover, an exhaust fan and an anion generator are disposed inside the formaldehyde removal filter cover, and negative ions generated by the anion generator and purified air are diffused to outside through the exhaust fan.

2. The externally-mounted and detachable air purification lamp fitting according to claim 1, wherein an external light emitting module is fixed on a bottom surface of the bottom cover, and a light emitting source is fixed in the external light emitting module.

3. The externally-mounted and detachable air purification lamp fitting according to claim 1, wherein the top of the breathable outer cover is provided with a mounting circular hole for fixing and mounting.

4. The externally-mounted and detachable air purification lamp fitting according to claim 3, wherein the formaldehyde removal filter cover is annular, an outer diameter of the formaldehyde removal filter cover is the same as an inner diameter of the breathable outer cover, an outer surface of the formaldehyde removal filter cover is attached to an inner wall of the breathable outer cover, and the exhaust fan and the anion generator are located in an inner ring of the formaldehyde removal filter cover.

5. The externally-mounted and detachable air purification lamp fitting according to claim 3, wherein the lamp is one of a ceiling lamp, a pendant lamp and a floor lamp.

6. The externally-mounted and detachable air purification lamp fitting according to claim 1, wherein the breathable outer cover is a cylindrical cover, and the formaldehyde removing filter cover is a circular ring cover.
